(19) United States
(12) Patent Application Publication
(10) Pub. No.: US 2009/0061478 A1
(43) Pub. Date: Mar. 5, 2009

(54) High-Speed Quantification of Antigen Specific T-Cells in Whole Blood by Flow Cytometry

(76) Inventors: Lene Have Poulsen, Hellerup (DK); Kivin Jacobsen, Hvalso (DK); Ian Storie, Madonas Rajons (LV); Jesper Laursen, Allerod (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/161,579

(22) PCT Filed: Jan. 30, 2007
(86) PCT No.: PCT/DK07/00045

§ 371 Date: Oct. 24, 2008

(30) Foreign Application Priority Data
Jan. 30, 2006 (US) 60/763339

(51) Int. Cl. C12Q 1/06

(52) U.S. Cl. 435/39

(57) ABSTRACT

The present invention discloses novel methods and compositions for identifying particular cell types in whole blood samples and defining either the concentration or ‘absolute count’ of these cells per unit volume of the sample. More specifically, the invention relates to methods for quantifying the antigen-specific T cells or defining the relative percentage of said cells in un-lysed whole blood. Further, the invention relates to kits for the preparation of whole blood samples for high-speed quantification of particular cell types, e.g. antigen specific T-cells, in said whole blood samples by flow cytometry.

### TECHNICAL FIELD

The present invention relates to methods and compositions for defining either the concentration or “absolute count” of particles, in particular cells, per unit volume. More specifically, the invention relates to methods for quantifying of antigen-specific T cells or defining the relative percentage of said T-cells in un-lysed whole blood. Further, the invention relates to kits for the preparation of a whole blood sample for high-speed quantification of antigen specific T-cells by flow cytometry.

### BACKGROUND OF INVENTION

There is a high interest in accurate, fast and reliable assays for quantification of antigen specific T cells. Enumeration of antigen specific T cells is important for measurement of and monitoring patients T cell responses to pathogens, autoantigens and tumor-derived antigens as well as for identification of transplanted patients at risk for viral infection (Heijnen IAFM et al. 2004). MHC reagents such as tetramers have previously been utilized for detection and enumeration of antigen specific T cells. On the flow cytometer platform, T-cell specific fluorescently labelled Major Histocompatability Complex (MHC) molecules loaded with the peptide of interest can be applied.

Heijnen IAFM et al. described a two step method for enumeration of cytomegalo virus (CMV) specific CD8+ T cells (Enumeration of Antigen-Specific CD8+ T Lymphocytes by Single-Platform, HLA Tetramer-Based Flow Cytometry: A European Multicenter Evaluation. Cytometry Part B (clinical cytometry) 62B:1-13, 2004). In this method Antigen specific T cells first were enumerated as a percentage of CD8+ T cells by a lyse/wash assay. Then the number of CD8+ cells/μL was enumerated by a lyse/no-wash method with the use of counting beads. This allowed for a calculation of the number of antigen specific CD8+ T cells per μL blood with sensitivity of 3-10 cells/μL. One of the disadvantages of this method is that it comprises two different preparations of blood cells and another is that the preparation time takes approximately 1 hour.

Another method for quantification of specific blood cells was described by Wölfl M et al. (Quantisation of MHC Tetramer-Positive Cells from Whole Blood: Evaluation of a Single-Platform, Six-Parameter Flow Cytometric Method. Cytometry Part A:120-130, 2004). In this method, antigen specific T cells were enumerated using only one tube in a lyse/no-wash assay, however, the total preparation time was relatively long and took more than 50 minutes. The detection limit for antigen specific T cells was reported to be 14 cells per 100 μL blood.

The two above cited prior studies are examples of the methods that utililize lysed blood cells. The lyse/no-wash assay typically requires approximately fifteen minutes incubation time followed by an additional fifteen minutes of lyse reaction time, after which the analysis is performed. The lyse/wash assay typically requires the same fifteen minutes incubation time (depending on temperature) and fifteen minutes of lyse reaction time as well as an additional 15-45 minute wash procedure prior to commencing the analysis. By contrast, no-lyse techniques generally only require approximately fifteen minutes of reaction time. Therefore, the use of no-lyse techniques could have the advantage that it may allow at least double the sample throughput at the preparation stage.

Another factor which can greatly improve the, speed, efficiency, safety, accuracy and reproducibility of clinical analytical results is the use of disposable containers containing pre-packaged, pre-measured or pre-mixed reagents or flow-cytometer counting beads. Disposable containers for absolute cell counting are known in the art. Such containers comprise dispensed portions or aliquots containing a known, fixed number of microparticles per tube, wherein the microparticles are utilized to calibrate the counting. Knowledge of the number of microparticles and, crucially, maintenance of this number within the tube during handling (e.g., prior to and during addition of the sample), is essential to the accuracy of the counts obtained.

It is known for example, to employ disposable containers containing a pre-determined number of microparticles. For example, a commercially available counting system comprises BD TruCOUNT Tubes (Catalog No. 340334, BD Biosciences San Jose, Calif.). The TruCOUNT absolute-count tubes contain a lyophilised pellet that dissolves during sample preparation, releasing a known number of fluorescent beads. The tube comprises a stainless steel retainer in the form of a grid which is positioned near the closed end of the tube and above the lyophilised pellet. The stainless steel retainer prevents the lyophilised pellet from falling out of the container during routine handling (such as for example, inversion or shaking of the tube), and accordingly maintains the fixed predetermined number of microparticles in the tube.

Nevertheless, problems remain with such embodiments. These generally arise from the fact that the lyophilisation results in a pellet, which is “fluffy” and easily breaks up from handling. Specifically, it is crucial to avoid disturbing the lyophilised pellet during sample handling and addition. Thus, the operating instructions for the TruCOUNT tubes specifically caution against disturbing the steel retainer and the pellet containing the beads, and advise pipetting above the stainless steel retainer. Furthermore, depending on the pitch of the stainless steel grid, it may not be totally effective in preventing portions of the lyophilised pellet from being detached and falling out of the tube. In order to minimise disturbance to the lyophilised pellet and grid, the TruCOUNT tubes have to be packaged in a protective pouch in a controlled atmosphere. Once the protecting pouch has been opened, the pellet will absorb moisture and consequently shrink. When this happens, the pellet is at risk of falling through the grid. Thus, when any of the tube, the pellet or grid is disturbed, or portions are lost, the absolute count obtained is potentially subject to error. The operating instructions for the TruCOUNT tubes state that the tubes should be discarded if the pellet has been disturbed in any manner.

Furthermore, methods of absolute counting which employ microbeads need to be carefully optimised to maintain the precision and consistency (i.e., count to count variability) of counts. One primary problem is that not all beads in the mixed sample may be counted, i.e., the actual number of beads counted with the flow cytometer is lower than expected from the predetermined number present in the tube. This arises due to the tendency of microparticles to adhere to one another to form doublets, triplets, quadruplets, etc. This is a particular problem with the beads used in the TruCOUNT tubes described above, in which (depending on batch) there is usually 5-10% multiple beads in a tube. Although this can be accounted for in the bead value quoted, it may give rise to discrepancies in the absolute counts.

In addition to adhering to one another, microparticles also tend to adhere to the surface of the container. Finally, some particles are not registered by the flow cytometer due to dead time in the sample acquisition. These factors compound to cause a discrepancy between the number of beads applied to the flow cytometer and the number of beads registered by the detectors of the flow cytometer in prior art methods.

A further problem is inter-count variability, i.e, the consistency of counts obtained through repeated processing. In other words, it is of the utmost importance that the variation in count between identical samples is low, i.e. you get the similar count every time a particular sample is measured. Here, the primary cause of the non-reproducibility appears to be microparticles adhering to the walls of the container in variable numbers.

The primary cause of this problem is the “stickiness” of the microparticles, i.e., the tendency of the microparticles to adhere to other components. This appears to be dependent on the nature of the material from which the microparticles are made, and the conditions in the environment in which they are counted. Variables such as pH, ionic strength, hydrophobicity and temperature of the sample medium can and do cause microparticles to have increased adhesiveness. Coating the walls of the container with, for example, BSA can reduce but not completely eliminate the problem. Multiplying the count obtained with a correction factor to account for the “lost” beads may also help reduce the discrepancy.

### SUMMARY OF INVENTION

With the capability of identifying, quantifying or sorting antigen specific T cells from the blood of patients without the need for methods such as application of lysing reagents that may harm or change the cells, the high-speed no-lyse analysis method disclosed herein provides a means for efficient counting or purification of functional antigen specific cells. Such purified cells may both be used for research purposes, diagnostics as well as for treatment. Purification of unperturbed cells may also be a pre-requisite for efficient and reliable ex-vivo expansion of antigen specific T cells.

Thus, the invention discloses novel methods and compositions which allow overcoming the problems inherent in the prior art discussed above.

The use of MCH Dextramers provides novel and advantageous features in the methods and compositions disclosed in accordance with the present invention. MHC Dextramers consist of a polymer backbone carrying an optimized number of peptide-loaded MHC and fluorochrome (FITC, RPE or APC) molecules. The MHC molecules are aligned as pearls on a string on the dextran backbone. Avidin-biotin bonds ascertain a firm anchoring of the MHC moieties to the dextran backbone that carries the fluorochromes. The MHC Dextramers are multimeric reagents that have an apparent higher T-cell receptor (TCR)-binding affinity compared with single MHCs. The MHC Dextramers expose TCRs to numerous peptide-loaded MHCs and the apparent higher binding affinity of the MHC Dextramers is caused by an increased avidity, which can be defined as the sum of the individual affinities of the multiple MHC and TCR interactions. It is possible to use combinations of MHC reagents with different specificities and/or fluorochrome labels. Furthermore, it is possible to use such combinations of MHC reagents together with combinations of antibody reagents. It is also possible to use combinations of MHC reagents, antibody reagents and counting beads in the described assay format. This allows for counting the number of cells of various subtypes of antigen specific T cells pr volume unit (e.g. μL) blood.

Other novel and advantageous features are provided by methods of the present invention wherein antibody reagents and/or microparticle counting beads, both used in analyses of antigen-specific T cells within un-lysed whole blood, are encapsulated within or embedded on or within an embedding medium, or “matrix”, as a means to retain the antibody reagents and/or microparticle counting beads within a container.

Further, because lyses of red blood cells are rendered obsolete by the assay disclosed in the method according to the invention, sample handling time may be shortened to approximately 5 minutes and thereby the total time of the assay may be shortened to 20-25 minutes.

Briefly describing the invention, the following aspects may be mentioned.

One aspect of the invention relates to a method for quantification of antigen specific T cells in un-lysed whole blood, comprising the steps:

(a) adding a sample of un-lysed whole blood to a reaction vessel;
(b) mixing a reagent containing MHC molecules with the un-lysed whole blood;
(c) mixing at least one antibody reagent with the un-lysed whole blood and reagent containing MHC molecules;
(d) incubating the mixture of un-lysed whole blood and reagents for a first pre-determined period of time;
(e) diluting the mixture of blood and reagents with an isotonic buffer, in the dilution range of 1:3 to 1:15;
(f) analyzing the mixture of blood, reagents and isotonic buffer on a flow cytometer analyzer and quantifying antigen specific T cells present in the whole blood sample.

Another aspect of the invention is a method for quantification of antigen specific T cells in un-lysed whole blood, comprising the steps:

(a) adding at least one antibody reagent to a reaction vessel;
(b) mixing a sample of un-lysed whole blood with the antibody reagents;
(c) mixing a reagent containing MHC molecules to the un-lysed whole blood and antibody reagents;
(d) incubating the mixture of un-lised whole blood and reagents for a pre-determined period of time.
(e) diluting the mixture of blood and reagents with an isotonic buffer, in the dilution range of 1:3 to 1:15;
(f) analyzing the mixture of blood, reagents and isotonic buffer on a flow cytometer analyzer and quantifying antigen specific T cells present in the whole blood sample.

Another aspect of the invention relates to a kit for preparing a sample of un-lysed whole blood for flow cytometric quantification of antigen-specific T cells, comprising
(a) a container; and
(b) a matrix adhered to at least one wall of the container comprising at least one antibody reagent disposed in or on the matrix, wherein the at least one antibody reagent comprises an antibody capable of binding to a chemical marker characteristic of a particular blood cell type.

Another aspect of the invention relates to a kit for preparing a sample of un-lysed whole blood for flow cytometric quantification of antigen-specific T cells, comprising
(a) a container; and
(b) a matrix adhered to at least one wall of the container comprising at least one MHC-molecule reagent disposed in or on the matrix, wherein the at least one MHC-molecule reagent comprises MHC molecules that comprise a peptide that enables binding of the peptide-MHC-molecule complex to the antigen-specific T-cells.

### DESCRIPTION OF DRAWINGS

FIGS. 1A-1C demonstrate flow charts for three methods for an assay for high-speed quantitation of antigen specific T cells in accordance with embodiments of the present invention.

FIG. 2A-2B show bivariate dot plots of flow-cytometry detection events obtained using the methods of the present invention, plotted as the logarithms of the compensated intensity of various fluorescent reporter molecules, of whole blood.

FIG. 2C shows a plot of the number counts detected from counting beads as a function of time; total bead count being obtained from Region R6 of FIG. 2A.

FIG. 3A-3B show bivariate dot plots of flow-cytometry detection events for whole blood samples obtained using the methods of the present invention, in which antibody reagents and MHC dextramer molecule reagents are embedded within a matrix.

FIG. 3C-3D shows bivariate dot plots of flow-cytometry detection events for whole blood samples obtained using the methods of the present invention, in which MHC dextramer molecules and antibody reagents are added to a vessel containing a whole blood sample.

### DESCRIPTION OF INVENTION

In this document, we disclose novel method(s) for high-speed flow cytometric quantification of antigen specific T-cells in whole blood. We show that MHC reagents and, particularly so-called MHC Dextramers, which are MHC reagents built with a dextran backbone, can be used for identification and single platform enumeration of antigen-specific cytotoxic T-cells, in particular cytomegalo virus (CMV) antigen specific T cells, in whole blood at detection levels below 1 cell per μL blood.

Before the present invention is described, it is to be understood that this invention is not limited to the particular embodiments described, as such methods, devices, and formulations may, of course, vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural referents unless the context clearly dictates otherwise, and includes reference to equivalent steps and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some preferred embodiments of the methods and materials of the invention are described below. All publications mentioned herein are incorporated herein by reference to disclose and describe the specific methods and/or materials in connection with which the publications are cited.

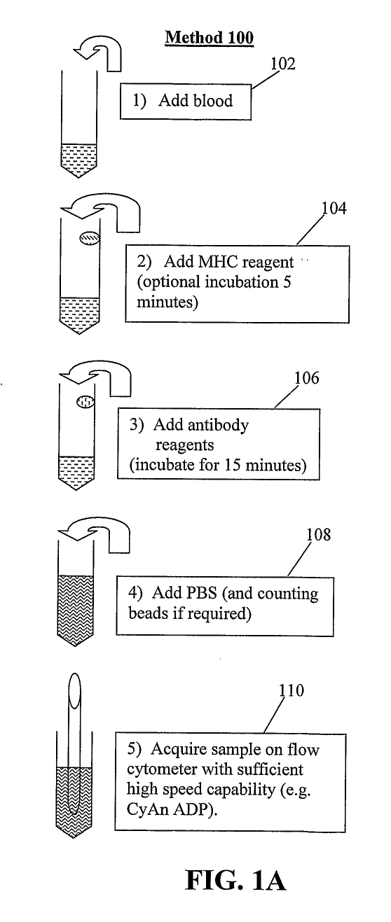

In conjunction with the following discussion the reader is referred to the appended FIGS. 1-3. FIG. 1A schematically illustrates a first embodiment of the method(s) of the invention termed herein “method 100”. FIG. 1B schematically illustrates a second embodiment of the method(s) of the invention, termed herein “method 200”. FIG. 1C schematically illustrates a third embodiment of the method(s) of the invention, thermed herein “method 300”. All three embodiments/methods are aimed for a high-speed quantification of antigen specific T-cells. By the “high-speed quantification” is meant that the quantification time does not exceed 30 min, preferably being 20-25 min.

### Method 100.

The following sequence of steps (FIG. 1A) illustrates a first method, termed “method 100”, in accordance with the present invention.
Step 102: Whole non-lysed blood is added to a reaction vessel.
Step 104: Reagents bearing MHC molecules (hereinafter referred to as “MHC reagents”), wherein the MHC molecules are preferably MHC Dextramers, are added to the non-lysed whole blood sample and, optionally, the MHC/blood mixture is incubated for 5 minutes.
Step 106: Appropriate antibody reagents are added to the reaction vessel containing the MHC/blood mixture and the mixture is incubated for 15 minutes.
Step 108: The reagent/blood mixture is diluted in the range of 1:3 to 1:15 (preferably 1:10) with an isotonic buffer such as phosphate buffered saline (PBS) solution and, optionally, counting beads are added.
Step 110. The sample is analyzed on a high speed flow cytometer analyzer, with the trigger set on a fluorescence parameter.

### Label/Reporter/Fluorescent Molecules

MHC reagents are, according to the invention, preferably represented by MHC Dextramers. Typically, MHC Dextramers comprise a polymer backbone, a dextran, carrying an optimized number of peptide-loaded MHC and fluorochrome (FITC, RPE or APC) molecules. The MHC molecules are aligned as pearls on a string on the dextran backbone. Avidin-biotin bonds ascertain a firm anchoring of the MHC moieties to the dextran backbone that carries the fluorochromes.

The MHC Dextramers are multimeric reagents that have an apparent higher T-cell receptor (TCR)-binding affinity compared with single MHCs. The MHC Dextramers expose TCRs to numerous peptide-loaded MHCs and the apparent higher binding affinity of the MHC Dextramers is caused by an increased avidity, which can be defined as the sum of the individual affinities of the multiple MHC and TCR interactions.

Antibody reagents according to the invention are represented by antibody molecules, which can recognize any antigens specific for T-cells. Non-limiting examples of such antibody reagents may be natural or recombinant full-length antibody molecules or antigen-binding fragments thereof specific for CD45, CD3, CD4, CD8 or the other antibody reagents discussed below.

Preferably, one or more of the antibody reagents and/or MHC molecule reagents are labelled with fluorescent reporter molecules, to enable the cell-binding agent and the cell to which it is bound, if any, to be identified and counted by flow cytometry analysis. Preferably, the microparticle counting beads are also labelled with a reporter molecule to enable counting.

Dyes having these properties may be selected from, but not limited to, the phycobiliproteins (especially phycoerythrin), fluorescein derivatives (such as fluorescein isothiocyanate), peridinin chlorophyll complex (such as described in U.S. Pat. No. 4,876,190), coumarin derivatives (such as aminomethyl coumarin), pthalocyanine dyes (such as Ultralite dyes (Ultradiagnostics)) and rhodamine derivatives (such as tetramethyl rhodamine or Texas Red (Molecular Probes)).

In some preferred embodiments fluorochromes may be selected from the group consisting of fluorescein isothiocyanate (FITC), phycoerythrin (PE), PE-Cy5, PE-Cy5.5, PE-Cy7, PE-A680, PE-TR (texas red), allophycocyanin (APC), APC-Cy7, Pacific Blue (PB), Cascade Yellow, Alexa dyes, coumarines or Q-dots.

Any one or more of these fluorochromes may be attached, preferably chemically conjugated, to the cell-binding agent such as an antibody or MHC molecule. Optionally, a fluorochrome (one or more than one) is disposed on or within the microparticle counting beads.

The majority of the fluorochromes may be conjugated with an antibody reagent by any method known in the art, e.g. reacting a maleimid-coupled fluorochrome with a thiolate-activated antibody, i.e. a chemoselective reaction, whereas FITC, Pacific Blue, Cascade Yellow, Cy5 and the Alexa dyes react directly with lysine amino-groups on the antibodies.

The reporter or “label” preferably comprises a light emitting detection means, and the light emitting detection means advantageously emits light of at least a fluorescent wavelength emission. It is preferred that the light emitting detection means comprises a fluorophore or fluorescent tag or group.

A “fluorescent tag” or “fluorescent group” refers to either a fluorophore or a fluorescent molecule or fluorescent protein or fluorescent fragment thereof. The fluorescent tag or group is such that it is capable of absorbing energy at a wavelength range and releasing energy at a wavelength range other than the absorbance range. The term “excitation wavelength” refers to the range of wavelengths at which a fluorophore absorbs energy. The term “emission wavelength” refers to the range of wavelength that the fluorophore releases energy or fluoresces. The term “fluorescent protein” refers to any protein which fluoresces when excited with appropriate electromagnetic radiation. This includes proteins whose amino acid sequences are either natural or engineered.

In some embodiments, the reporter label, preferably fluorescent tag, of the microparticle counting beads is different from that of the antibody and MHC molecule reagents. Preferably, the reporter labels are chosen such that the emission wavelength spectrum of one is distinguishable from the excitation wavelength spectrum of the other. The different reporter labels may be excitable by the same wavelength of light or different wavelengths. Preferably, the emission wavelengths are different. Alternatively, if the decay times of the excited species are different, time resolved fluorescence could be used.

In such an arrangement, it is possible to count the microparticle counting beads separately from the labeled reagents (i.e., the cells to which they are bound), for example, using a different fluorescent channel. However, while distinguishable reporter labels are preferred, it will be clear that this is not absolutely necessary. Indeed, in some embodiments, microparticle counting beads which are not labeled with fluorescent tags may be employed, while still being distinguishable from the labeled cells using other parameters. For example, the microparticle counting beads maybe distinguishable form the labeled cells either by size (scatter parameters), emission wavelength (fluorescence parameters) or fluorescence intensity.

In one preferred embodiment the fluorochromes or fluorophores may comprise fluorescein and tetramethylrhodamine or another suitable pair. In another preferred embodiment, the label may comprise two different fluorescent proteins. Fluorescent protein may be selected from the group consisting of green fluorescent protein (GFP), blue fluorescent protein, red fluorescent protein and other engineered forms of GFP.

Preferably, the polypeptide comprises a cysteine or lysine amino acid through which the label is attached via a covalent bond.

A non-limiting list of chemical fluorophores and fluorochromes suitable for use, along with their excitation and emission wavelengths, is presented in Table 1.

TABLE 1
Excitation and emission wavelengths of some fluorophores and
fluorochromes

| Fluorophore | Excitation (nm) | Emission (nm) |
|---|---|---|
| PKH2 | 490 | 504 |
| PKH67 | 490 | 502 |
| Fluorescein (FITC) | 495 | 525 |
| Hoechst 33258 | 360 | 470 |
| R-Phycoerythrin (PE) | 488 | 578 |
| Rhodamine (TRITC) | 552 | 570 |
| Quantum Red | 488 | 670 |
| PKH26 | 551 | 567 |
| Texas Red | 596 | 620 |
| Cy3 | 552 | 570 |
| Pacific Blue (PB) | 410 | 455 |

Examples of fluorescent molecules which vary among themselves in excitation and emission maxima may be selected from the list of Table 1 of WO 97/28261 (incorporated herein by reference). These (each followed by [excitation max./emission max.] wavelengths expressed in nanometers) include wild-type Green Fluorescent Protein [395(475)/508] and the cloned mutant of Green Fluorescent Protein variants P4 [383/447], P4-3 [381/445], W7 [433(453)/475(501)], W2 [432(453)/480], S65T [489/511], P4-1 [504(396)/480], S65A [471/504], S65C [479/507], S65L [484/510], Y66F [360/442], Y66W [458/480], I0c [513/527], W1B [432(453)/476(503)], Emerald [487/508] and Sapphire [395/511]. This list is not exhaustive of fluorescent proteins known in the art; additional examples may be found in the Genbank and SwissProt public databases.

The fluorescence of the microparticle counting beads must be such that it is sufficiently greater than noise from background in one fluorescence channel so as to be distinguishable from the reporter molecules bound to the reagents, and it is also distinguishable in other fluorescence channel(s). The term “sufficient” refers to one log difference between the dye(s) and the microparticle fluorescence. The concentration of the microparticle counting beads should be greater than or equal to the number of cells to be counted. Generally, a final bead count of at least 1000 beads per μl is preferred.

### Method 200

Referring now to FIG. 1B, the following sequence of steps illustrates a second method, termed “method 200”, in accordance with the present invention. The method 200 employs a pre-packaged and, optionally, disposable container 250 which also serves as the reaction vessel.
Step 202: A pre-packaged container 250 is provided, the container containing antibody reagents and, optionally, counting beads,
Step 204: Whole non-lysed blood is added to the reaction vessel.
Step 206: MHC reagents, wherein the MHC molecules are preferably MHC Dextramers, are added to the reaction vessel and the mixture is incubated for 15 minutes.
Step 208: The reagent/blood mixture is diluted in the range of 1:3 to 1:15 (preferably 1:10) with an isotonic buffer such as PBS.
Step 210. The sample is analyzed on a flow cytometer analyzer, with the trigger set on a fluorescence parameter.

### Matrix Material Utilized in the Pre-Packaged Container:

The pre-packaged container 250, which is also the reaction vessel of method 200, preferably contains a matrix material to retain and immobilize the reagents and/or microparticle counting beads prior to use. The matrix is such that it retains the microparticles in the container when dry but releases the microparticles into the sample medium when a sample containing cells of interest is added to the container. Preferably, the matrix dissolves in the sample medium to effect release. For this purpose, the matrix preferably comprises a gelatinous or viscous material, which may be liquid, semi-solid or gel-like in consistency. Preferably, the matrix is a viscous liquid.

The matrix may be substantially free of water, or it may comprise water. In one preferred embodiment, despite of appearing dry, the matrix contains some water, preferably the matrix comprises less than 30% of water, such as between 1% and 29%, for example between 5% and 25%, or such as between 10% or 25%, for example around 15%. In another preferred embodiment the matrix is preferably substantially free of water, such as the matrix comprising less than 10% of water. In some embodiments the matrix may also comprise a liquid other than water, such as glycerol, ethylene glycol, propylene glycol or others.

In various embodiments, it may be preferred the matrix that has a viscosity of 103 cP, in other embodiments a preferred matrix may have viscosity of 104 cP or 105 cP. The matrixes having viscosity of 106 cP or more are more preferred. The term “viscosity” refers to both/either dynamic viscosity and/or kinematic viscosity, which is/are preferably measured at a temperature of 25 degrees Celsius.

The matrix may be represented by a single contiguous mass, or it may be attached to the container as a number of separate pieces. Preferably it is contiguous. Preferably, however, the matrix is such that during handling or storage no portion of the matrix effectively detaches from the container to cause loss of microparticle counting beads.

According to the invention it is preferred that the matrix is water soluble, preferably readily soluble in aqueous media. Preferably, the matrix dissolves when a sample containing the cells of interest is added into a container comprising the matrix, or otherwise breaks up in such a manner as to release the microparticles into the sample medium. Preferably, all or substantially all of the microparticles are released into the sample medium.

The matrix may be present in any suitable quantity in a container. Preferably, the amount of matrix is sufficient to hold the required number of microparticles in the container. In various embodiments, the amount of matrix varies from about 100 mg to about 1 mg. In some preferred embodiments the amount of matrix is less than 100 mg, such as less than 50 mg, preferably less than 30 mg, such as less than 20 mg, preferably less than 10 mg. More preferred when the matrix is present at an amount of not more than 10 mg, preferably less than 5 mg, or even more preferably 3 mg or less than 3 mg.

Preferably, the matrix comprises an environment that is neither oxidizing nor reducing in order to avoid any unwanted redox-reactions. For example, if a carbohydrate matrix (such as described below) is employed, the carbohydrates are preferably non-reducing. The matrix is preferably composed of compounds that do not crystallise, crack or change phase at any temperature so that it may be transported and stored under normal (standard) conditions. It is preferable to use a matrix with low melting point to avoid the crystallization. A high molecular weight matrix may be preferred to reduce the osmotic effect on the sample preparation.

In some preferred embodiments, the matrix may be based on a water soluble sugar mixture. The matrix or embedding medium may comprise one or more compounds including carbohydrates, polymers, small proteins or others. Examples of suitable carbohydrates for use in a matrix include, but not limited to, saccharose, arabinose, ribulose, fructose, sorbose, glucose, mannose, gulose, galactose, sucrose, lactose, maltose, trehalose, raffinose and melizitose. Cellulose as well as carboxylated or otherwise derivatised cellulose products may also be employed. Examples of suitable polymers for use in a matrix include, but not limited to, polyvinyalcohols, polyethylene glycols, polyethylene imines, polyacryl amides, polyaziridines, glycols, polyacrylic acids, esters or derivatives thereof. A block co-polymers of the aforementioned could also be used. Examples of small proteins include BSA other albumins or protein fragments such as Byco A. Mixtures of two or more of the latter may also be used. The components of the matrix may be present in any suitable proportion consistent with the desirable properties outlined above.

Specifically, we disclose matrices comprising mixtures of carbohydrates, for example, fructose, trehalose and raffinose. The matrix according to the embodiments of the invention may comprise any two of fructose, trehalose and raffinose at any ratio, preferably at 2:1, 1:1 or 1:2 ratio. The matrix may comprise 2:1, 1:1 or 1:2 of fructose and trehalose, in particular one preferred embodiment relates to 3 mg of a 1:1 mixture of fructose and trehalose.

The matrix may also perform other functions, such as for example providing a stable and inert medium for preserving the microparticles during storage. For this purpose, other components may also be additionally included. These may include any one or more of preservatives, detergents, fixatives, antioxidants and pH-stabilizers. Examples of preservatives include bronidix, sodium azide and thiomersal. Examples of detergents include Tween, Triton, Brij, Pluronic and Tetronic as well as derivatives and mixtures of the aforementioned. Examples of fixatives include vinylsulfone and glutaraldehyde. The matrix may comprise one or more antioxidants, which are molecules that are radical scavengers. The radicals can be O-, N- C- or S-radicals. In some embodiments, the matrix may comprise scavengers for oxygen-derived radicals such as the superoxide anion or the hydroxyl radical formed by atmospheric oxygen under influence of light, heat or other environmental factors. Examples of such radical scavengers may be ascorbic acid, beta-carotene, bilirubin, butylated hydroxytoluene (BHT), butylated hydroxyanisol (BHA) tert-butylhydroquinone (TBHQ) d-alpha-tocopherol, trolox and hydroxyanisol. Examples of pH-stabilizers include Good buffers, HEPES, MES, phosphate, citrate.

The following are two exemplary methods which may be used for the preparation of matrixes for the purposes of the invention:

### First Method

1. 20% (w/v) solutions of the sugars are made up and mixed in a 1:1 ratio.
2. 15 μL of the mixture is added to 5 mL Falcon tubes (Becton Dickinson).
3. If desired, antibodies are added in the required amount.
4. CytoCount™ beads are added to each tube using reverse pipetting in the required amount.
5. The mixtures are dried under vacuum at room temperature over night and are subsequently stored at 2-8° C. protected from light until used.

### Second Method

1. 20% (w/v) solutions of the sugars are made up and mixed in a 1:1 ratio.
2. 15 μL of the mixture is added to 5 mL Falcon tubes (Becton Dickinson).
3. Antibodies are added in the required amount.
4. If desired, an antioxidant is added in the required amount.
5. CytoCount™ beads are added to each tube using reverse pipetting in the required amount.
6. The mixtures are dried under vacuum at 2-8° C. over night and are subsequently stored at 2-8° C. protected from light until used.

### Microparticle Counting Beads

In general, the microparticle counting beads are particles with scatter properties that put them in the context of the cells of interest when registered by a flow cytometer. They can be either labelled with antibodies, fluorochromes or other small molecules or they may be unlabelled. In some embodiments of the invention, the beads may be polystyrene beads with molecules embedded in the polymer that are fluorescent in most channels of the flow-cytometer.

The microparticle counting beads employed in the methods and compositions described herein are small, preferably between 0.1 μm and 100 μm in diameter, such as between 0.5 μm and 50 μm or between 1 μm and 10 μm. In some preferred embodiments the size of the microparticle beads may preferably be about 5 μm in diameter. Generally, the microparticles preferably are made of such material and are of such size as to stay suspended, with minimal agitation if necessary, in solution or suspension (i.e., once the sample is added). The microparticle beads do preferably not settle any faster than the cells of interest in the sample. The material from which the microparticles are made preferably of such quality and composition as to avoid clumping or aggregation of the beads, i.e., the formation of doublets, triplets, quadruplets, etc. A final count of 1000 microparticles per μl of blood is preferred.

The microparticles may be, or in some embodiments are preferably, labeled with a reporter molecule, such as a fluorescent molecule (which is selected from described herein). Alternatively, or in addition, an autofluorescent microparticle may be employed.

Microparticles may be selected from the group consisting of fixed chicken red blood cells, coumarin beads, liposomes containing a fluorescent dye, fluorescein beads, rhodamine beads, fixed fluorescent cells, fluorescent cell nuclei, microorganisms and other beads tagged with a fluorescent dye. However, preferred examples of compact particles include microbeads, such as agarose beads, polyacrylamide beads, polystyrene beads, silica gel beads, etc. Beads or microbeads suitable for use may include those which are used for gel chromatography, for example, gel filtration media such as Sephadex. Suitable microbeads of this sort include Sephadex G-10 having a bead size of 40-120μ (Sigma Aldrich catalogue number 27,103-9), Sephadex G-15 having a bead size of 40-120 μm (Sigma Aldrich catalogue number 27,104-7), Sephadex G-25 having a bead size of 20-50 μm (Sigma Aldrich catalogue number 27,106-3), Sephadex G-25 having a bead size of 20-80 μm (Sigma Aldrich catalogue number 27,107-1), Sephadex G-25 having a bead size of 50-150 μm (Sigma Aldrich catalogue number 27,109-8), Sephadex G-25 having a bead size of 100-300 μm (Sigma Aldrich catalogue number 27,110-1), Sephadex G-50 having a bead size of 20-50 cm (Sigma Aldrich catalogue number 27,112-8), Sephadex G-50 having a bead size of 20-80 μm (Sigma Aldrich catalogue number 27,113-6), Sephadex G-50 having a bead size of 50-150 μm (Sigma Aldrich catalogue number 27,114-4), Sephadex G-50 having a bead size of 100-300 μm (Sigma Aldrich catalogue number 27,115-2), Sephadex G-75 having a bead size of 20-50 μm (Sigma Aldrich catalogue number 27,116-0), Sephadex G-75 having a bead size of 40-120 μm (Sigma Aldrich catalogue number 27,117-9), Sephadex G-100 having a bead size of 20-50 μm (Sigma Aldrich catalogue number 27,118-7), Sephadex G-100 having a bead size of 40-120 μm (Sigma Aldrich catalogue number 27,119-5), Sephadex G-150 having a bead size of 40-120 μm (Sigma Aldrich catalogue number 27,121-7), and Sephadex G-200 having a bead size of 40-120 μm (Sigma Aldrich catalogue number 27,123-3). Sepharose beads, for example, as used in liquid chromatography, may also be used. Examples such beads may be Q-Sepharose, S-Sepharose and SP-Sepharose beads, available for example from Amersham Biosciences Europe GmbH (Freiburg, Germany) as Q Sepharose XL (catalogue number 17-5072-01), Q Sepharose XL (catalogue number 17-5072-04), Q Sepharose XL (catalogue number 17-5072-60), SP Sepharose XL (catalogue number 17-5073-01), SP Sepharose XL (catalogue number 17-5073-04) and SP Sepharose XL (catalogue number 117-5073-60) etc.

Other preferred particles for use in the methods and compositions described here are those microparticles that comprise plastic microbeads. Although plastic microbeads are usually solid, they may also be hollow inside and could be vesicles and other microcarriers. Plastic materials such as polystyrene, polyacrylamide and other latex materials may be employed for fabricating the beads, but other plastic materials such as polyvinyl chloride, polypropylene and alike may also be used. Polystyrene is a preferred material. The microparticles include unlabelled beads, beads with antibodies, fluorochromes or other small molecules conjugated to the surface or beads with fluorochromes embedded in the polymer.

### Method 300

Referring now to FIG. 1C, the following sequence of steps illustrates a third method, termed “method 300”, in accordance with the present invention. The method 300 employs a pre-packaged and, optionally, disposable container 250 which may be similar to that described above in connection with the method 200. As described previously, the pre-packaged container 250 also serves as the reaction vessel and, preferably, contains a matrix material to retain and immobilize the reagents and/or microparticle counting beads prior to use.
Step 302: A pre-packaged container 250 is provided, the container containing antibody reagents, MHC molecule reagents and, optionally, counting beads.
Step 304: Whole non-lysed blood is added to the reaction vessel.
Step 306: The mixture is incubated for 15 minutes.
Step 308: The reagent/blood mixture is diluted in the range of 1:3 to 1:15 (preferably 1:10) with an isotonic buffer such as PBS.
Step 310. The sample is analyzed using a flow cytometer analyzer, with the trigger set on a fluorescence parameter.

The primary difference between the method 200, illustrated in FIG. 1B, and the method 300, illustrated in FIG. 1C, is that the MHC molecule reagents are provided pre-packaged within the container 250.

Thus, the present invention describes a novel method for quantification of antigen specific T cells in un-lysed whole blood which may be used in the three above described assay formats (referred above as method 1, method 2 and method 3).

In a first embodiment (embodiment 1) the method of the invention may comprises the steps of:
(a) adding un-lysed whole blood to a reaction vessel;
(b) mixing a reagent containing MHC molecules with the un-lysed whole blood;
(c) mixing at least one antibody reagent with the un-lysed whole blood and reagent containing MHC molecules;
(d) incubating the mixture of blood and reagents for a first pre-determined period of time;
(e) diluting the mixture of blood and reagents with an isotonic buffer, in the dilution range of 1:3 to 1:15;
(f) analyzing the mixture of blood, reagents and isotonic buffer on a flow cytometer analyzer and quantifying of antigen specific T cells present in the whole blood sample.

The above embodiment of the method may further comprise the step of incubating the mixture of whole blood and reagents containing MHC molecules for a second pre-determined period of time following the step of mixing reagents containing MHC molecules (b).

The total time for preparation of the blood sample for quantification of T-cells according to the above embodiment is about 30 min, such as 25-27 min. This period of time includes the time of incubation of blood sample with a MHC reagent and antibody reagent of step (d), which according to the invention do not exceed 20 min, preferably being 15 min (this incubation time referred herein as “first pre-determined period of time”). Further, it includes the time of optional incubation of a blood sample with a MHC reagent which may follow the step (b) (this incubation time referred herein as “second pre-determined period of time”). The second predetermined period of time according to the invention is or about 5 min. Incubation of MHC reagent(s) after adding antibody reagent(s) is suitable provided that none of the antibody reagents blocks the binding of the MHC-bearing reagent(s) to T-cell receptor sites. Some CD8 antibody reagents, and others, from certain clone lines are known to block the binding of the MHC molecules to T-cells and, if these are used, these antibody reagent(s) should be added and incubated after addition of the MHC-molecule reagents.

The mixture of blood and the reagent is diluted with an isotonic buffer, such as for example phosphate buffered saline (PBS), in the dilution range 1:3 to 1:15. In one embodiment a preferred dilution range may be 1:10, however in the other embodiments a preferred dilution range may be different.

In some preferred embodiments the method may further comprise the step of adding counting beads to the mixture of blood, reagents and isotonic buffer which preferably follows the diluting step (e). Although counting beads can be added at any time, it is preferable to add them just prior to running the cytometer analysis, as this allows less time for clumping or settling of the beads.

Analyzing the mixture of blood, reagents and isotonic buffer for concentration for the presence of antigen specific T cells in the whole blood sample is made using a flow cytometer analyzer. According to the invention the flow cytometer trigger is set to a fluorescence parameter. A flow rate through the flow cytometer analyzer according to the invention is preferably between 50 and 200 μL/min

In some preferred embodiments an MHC molecule and/or an antibody reagent may be labelled with a fluorochrome. It may be preferred that an MHC molecule is labelled with a first fluorochrome and an antibody is labelled with a second fluorochrome. Further, it may be preferred that an MHC molecule comprises a dextran backbone.

In another embodiment (embodiment 2) the method for quantification of antigen specific T cells in un-lysed whole blood may according to the invention comprise in the steps of:
(a) adding at least one antibody reagent to a reaction vessel;
(b) mixing un-lysed whole blood with the at least one antibody reagent;
(c) mixing a reagent containing MHC molecules to the un-lysed whole blood and antibody reagents;
(d) incubating the mixture of blood and reagents for a pre-determined period of time.
(e) diluting the mixture of blood and reagents with an isotonic buffer, in the dilution range of 1:3 to 1:15;
(f) analyzing the mixture of blood, reagents and isotonic buffer on a flow cytometer analyzer and quantifying of antigen specific T cells present in the whole blood sample.

According to this embodiment of the method the pre-determined period of time is approximately fifteen minutes, and the total time for preparation of the blood sample for T-cell quantification, thus, may be less than 20 min.

The mixture of blood and the reagent is diluted with an isotonic buffer, such as for example phosphate buffered saline (PBS), in the dilution range 1:3 to 1:15. In one embodiment a preferred dilution range may be 1:10, however in the other embodiments a preferred dilution range may be different.

The method of above (embodiment 2) may further comprise the step of adding counting beads to the antibody reagents, wherein said step. Analyzing the mixture of blood, reagents and isotonic buffer for the presence of antigen specific T cells in the whole blood sample is made using a flow cytometer analyzer. According to the invention the flow cytometer trigger is set to a fluorescence parameter. A flow rate through the flow cytometer analyzer according to the invention is preferably between 50 and 200 μL/min. In some preferred embodiments of the method an MHC molecule and/or an antibody reagent may be labelled with a fluorochrome. It may be preferred that an MHC molecule is labelled with a first fluorochrome and an antibody is labelled with a second fluorochrome. Further, it may be preferred that an MHC molecule comprises a dextran backbone.

In both embodiments described above when referred to “at least one antibody reagent” is meant that it may be used one or more antibody reagents, wherein the wording “more antibody reagents” means that the antibody reagent(s) may be represent by a mixture of different antibodies. Embodiments of the antibody reagent(s) are discussed in the above and below sections of the instant application.

### Kits

We further disclose kits for absolute counting which use or include the methods and compositions described here. Such a kit is used for preparing an un-lysed whole blood sample for high-speed flow cytometric quantification of antigen-specific T cells as described above. In general, the kit can comprise a pre-packaged container initially provided with the components or compositions described here, namely, the matrix, microparticles, the antibody reagents and, optionally (as in the method 300) MHC molecule reagents. The kit may comprise packaging, such as sealed packaging, and it may further comprise instructions for use. Provision of such kits, to users, can expedite and simplify the analytical procedures as conducted, for instance, in a clinical laboratory, thereby leading to greater analytical throughput and reproducibility, and reducing user errors.

In particular, the invention in one embodiment relates to a kit for preparing an un-lysed whole blood sample for flow cytometric quantification of antigen-specific T cells which comprises:
(a) at least one container, and
(b) a matrix adhered to the at least one wall of the container comprising at least one antibody reagent disposed in or on the matrix, wherein the at least one antibody reagent comprises an antibody capable of binding to a chemical marker characteristic of a particular blood cell type.

In another embodiment, the invention relates to a kit for preparing an un-lysed whole blood sample for flow cytometric quantification of antigen-specific T cells which comprises:
a) a container; and
(b) a matrix adhered to at least one wall of the container comprising at least one MHC-molecule reagent disposed in or on the matrix, wherein the at least one MHC-molecule reagent comprises MHC molecules that comprise a peptide that enables binding of the peptide-MHC-molecule complex to the antigen-specific T-cells.

For ease of handling, the matrix according to the invention is preferably comprised by the container, where it is adhered to at least one wall of the container. The container can take any suitable form and be made of any suitable material and may be included within a kit.

The container may in particular take the form of a reagent tube, such as a test tube, or microtitre plate or strips for a microtitre format. Where microtitre plates are used as the container, each of the cell-binding agents, reporters, and microparticles in each of the plates may be the same, or different.

Preferably, the container has a tubular or elongate shape. In some embodiments, the container has a non-circular cross section, for example, a square cross section or a triangular cross section or a polygonal cross section. However, in various embodiments, the container has a circular cross section, and is preferably cylindrical in shape.

The container is preferably closed at one end, and preferably the matrix comprising in which the microparticles are disposed is positioned at or towards the closed end. The closed end may be flat or have a bowl shape. The microparticles are effectively retained in the container during handling through the matrix, and there is therefore no requirement for a retaining grid. Preferably, therefore, the container does not comprise such a grid. The container may, however be closed by a Hd or a top seal, e.g. plastic foil (preferred for microtitre plates) or wax or oil to prevent contact with air or moisture. The air in the container could be filtered air, neutral gases, carbon dioxide or any gas that has a protective effect on the reagents in the container. It should be clear that the sample could be added without removing the top seal. Furthermore, a top seal will make it possible to include many different reagent mixtures in one microtitre plate and to use the desired reagent mixtures by simply puncturing the seal, leaving the unused mixtures undisturbed.

The container is preferably transparent or translucent (e.g., frosted) in at least one portion, preferably over the whole of the container. It may however also be impervious to light in order to protect the contents from light. A transparent container may also be packed in a secondary container that is impervious to light, thus protecting the contents from light. The secondary container may be made of foil, preferably a foil bag or pouch. It may also be a box made of plastic or any other material that is or can be made impervious to light.

The primary container may be made of any suitable material, such as glass, heat resistant glass (e.g., Pyrex glass), plastic, polypropylene, polystyrene, etc. Preferably, the material from which the container is made is inert and resistant to chemical attack.

At least a portion of the inner surface of the container, preferably at least one or more of the walls of the container may be treated, by for example coating. The coating may comprise for example, a hydrophobic material such as silicone, or a material capable of preventing components of the sample from sticking to the container. It may also preferably prevent the microparticles (when suspended after addition of sample) from sticking to the surfaces of the container. Such coating material may comprise for example, proteins such as bovine serum albumin (BSA), casein or gelatine. Coating in such a manner with blocking agents prevents non-specific binding to the container.

The container may also comprise a mixing device, preferably incorporated in the container, for mixing the sample or other reagents. Suitable mixing devices comprise vibrating chips or magnets.

The container may be labelled with a means of identification. These may comprise barcodes, infoglyphs or chips, preferably RFID chips. The means of identification may also be capable of storing other information. Such other information may comprise any one or more of the following: patient identification or information, information on the sample, information on the reagents (e.g., manufacture date, lot number, correct protocol), information on steps the sample has been submitted to (e.g., incubation time, temperature, any waiting time between steps, etc).

Preferred containers are those which are employed for laboratory purposes, in particular, for flow cytometry.

A kit according to the invention comprises a matrix adhered to the at least one wall of the container comprising at least one antibody reagent disposed in or on the matrix, wherein the at least one antibody reagent comprises an antibody capable of binding to a chemical marker characteristic of a particular blood cell type. Embodiments of the matrix comprised by a kit of the invention are discussed in detail above. In particular embodiments the kit comprises a matrix which is a polymer, for example protein (for other example see above discussion), which is capable to adhere to at least one wall of the container. comprises a carbohydrate. Preferably, the matrix comprises a carbohydrate, preferably the carbohydrate is a sugar or a mixture of sugars. The choice of sugars comprised by the matrix is discussed above.

The matrix comprises at least one antibody reagent disposed in or on the matrix, wherein the at least one antibody reagent comprises an antibody capable of binding to a marker characteristic of a particular blood cell type. According to the invention the antibody reagent(s) is (are) present (disposed) in or on the matrix in a quantity of from 0.02 to 4 μg (each antibody reagent) per 100 μl of un-lysed whole blood sample, such as from 0.05 μg to 3 μg, for example between 1 μg and 2 μg per 100 microliters of the sample.

The antibody reagent comprises an antibody or an antibody fragment which is capable of specifically binding to a chemical marker (antigen) characteristic for a particular blood cell type. The chemical marker may be any known cell marker and the choice of this marker is dependent on the type of cells which is going to be defined and counted using the methods described above. For example for counting antigen specific T-cells use an antibody reagent comprising an antibody to CD3, CD45, CD8 could be advantageous. An antibody may be any antibody molecule or a fragment thereof prepared by a any method well-known in the art or obtained form a commercial manufacturer. It is preferred that at least one antibody reagent of the kit is labelled with a fluorochrome. Examples of fluorochromes are discussed above.

The kit in some embodiments may further comprise an MHC-molecule reagent which is disposed in or on the matrix, wherein the MHC-molecule reagent comprises MHC-molecules that comprise a peptide that enables binding of the peptide-MHC-molecule complex to the antigen specific T-cells. An example of such a peptide is NLVPMVATV from human cytomegalo virus PP65 structural protein.

The MHC-molecule reagent according to the invention may also comprise a dextran backbone.

According to the invention the MHC-molecule reagent is present in or on the matrix in a quantity of from 0.3 to 30 μl, such as for example about 1 μl, about 5 μl or between about 10 μl and about 25 μl, per 100 microliters of un-lysed whole blood sample.

A kit according to the invention may comprise microparticles which may comprise polystyrene, latex, agarose or acrylamide beads. Microparticles of the invention are discussed in detail in the above sections.

The reagents included in a kit according to the invention may comprise any combination of
1) at least one antibody reagent disposed in or on the matrix or provided in a separate container (or containers), wherein the at least one antibody reagent comprises an antibody capable of binding to a chemical marker characteristic of a particular blood cell type, and
2) at least one MHC-molecule reagent disposed in or on the matrix or provided in a separate container (or containers), wherein the MHC-molecule reagent comprises a peptide that enables binding of the peptide-MHC-molecule complex to the antigen-specific T-cells.

The kit may include one or more containers, each having a matrix adhered to the container which comprises at least one antibody reagent or MHC molecule reagent disposed in or on the matrix, such as the kit which comprises a second container which, depending on different embodiments, comprises a matrix comprising at least one MHC-molecule reagent or one or more antibody reagents described herein, wherein the matrix or antibody reagent(s) are present adhered to at least one wall of this second container. Further, the kit may include one or more separate containers of purified antibody reagent(s) or MHC molecule reagent(s). Preferably, a kit according to the invention further comprises instructions for use.

For additional details of the composition, manufacture, handling and use of the matrix and associated containers, materials, packaging and kits, the reader is referred to international patent application publication WO 2006/090283 A2, titled “Cell Counting” and published on 31 Aug. 2006, said international patent application publication hereby incorporated by reference herein in its entirety.

### EXAMPLES

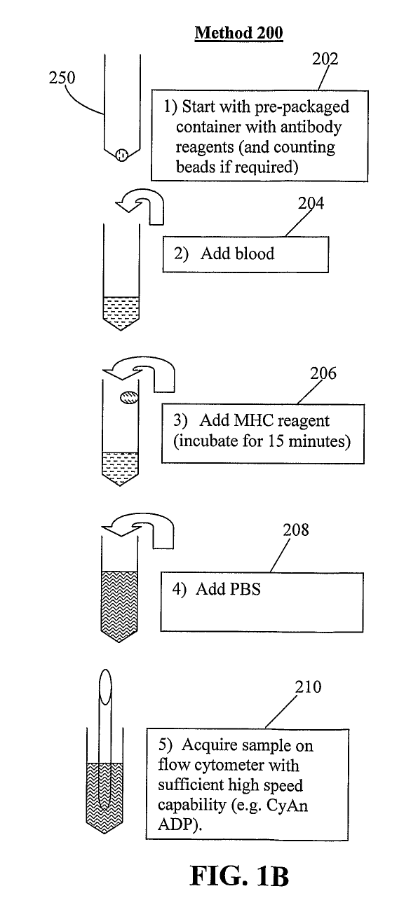

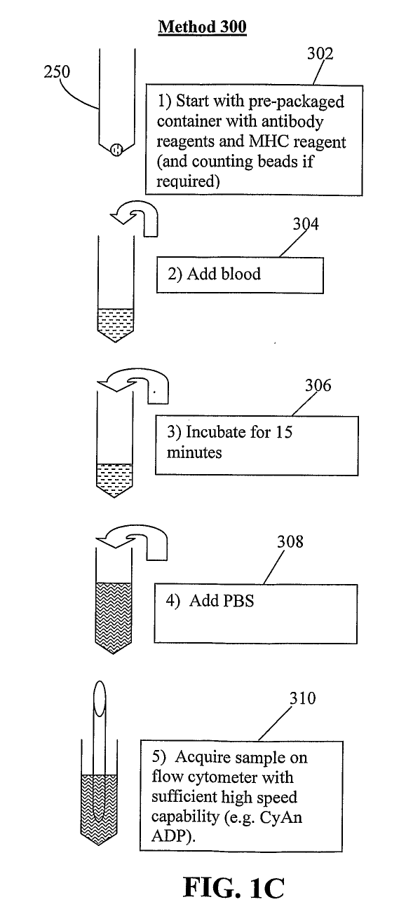

Some examples of the evaluation and practice of the present invention are now presented. It is to be understood that these examples are provided to illustrate some of the benefits and uses of the present invention and are not intended to be exhaustive or limiting of the invention in any way. In our own study employing the above methods, analyses were performed on a CyAn ADP (manufactured by Dako, Glostrup Denmark) flow cytometer. Acquisition time was typically 5 minutes for 20.000 CD8+ events. Acquisition rates were between 50 and 200 μL/min. The method relies on fluorescence parameters, thus the trigger is set on a fluorescence parameter. As an example, a fluorochrome labelled CD45 specific antibody reagent can be used to set the trigger discriminator to allow the flow cytometer to distinguish between red blood corpuscles and stained white blood cells. FIGS. 2A-2C shows flow cytometry results, obtained according to the methods of the present invention, of whole blood stained with Mouse Anti-Human CD45/Pacific Blue, CD3/FITC, CD8/R-PE and MHC Dextramer (CMV)/APC reagents. After 15 min incubation at room temperature CytoCount™ beads and PBS were added. Subsequently, the samples were analyzed on a CyAn ADP flow cytometer analyzer, in which counts were obtained simultaneously on multiple channels of the various events of interest—fluorescence from the various fluorophore or fluorochrome labels and from counting beads as well as scatter parameters. Results were analyzed both automatically and graphically.

In general, the emitted light that is detected in a flow cytometer analyzer can provide information relating to distinctive spectral signatures of cells as they pass through the sensing region. The number of detection events for each of the various distinctive spectral signatures is then related to the number of cells associated with each such signature. The characteristics of the detected light that is scattered and emitted from the cells as they pass through the sensing region may, in general, be stored in computer memory for later offline analysis. The stored data are generally in a format known as “list mode” in which the data collected for each cell comprises a “recorded event” comprising several parameters. The stored list mode data comprises a data value for each measured parameter relating to the first detected cell, to the second detected cell, and to each subsequently detected cell, in the sequence obtained, up until the data for the final detected cell. The stored parameters for each cell generally relate to the level of detection of light scattered by the cell or light emitted from the cell within certain wavelength bands of interest.

The data contained within a list mode file based on n (n an integer) cytometrically determined parameters generally defines clusters of cells, within an n-dimensional analytical space, having particular scattering or emission properties. The process of analytically discriminating among and between cells having differing spectral characteristics and separating the cells into different populations based upon these characteristics is known as gating. By a combination of graphical and statistical analysis, partial discriminations and selection of progressively refined data subsets may be made based upon fewer than the full set of parameters, using projections of the data onto graphs (such as conventional bivariate plots, also called dot plots) illustrating the distribution of spectral characteristics within restricted subspaces. The resulting data subsets may then be separately analyzed, either statistically or graphically, using the values of the remaining or other parameters.

In general, any target sub-population of cells, such as antigen-specific cytotoxic T-cells in the present example, will test positive for a certain set of biochemical markers. However, each such biochemical marker may be associated with a variety of cell types, other than the particular target sub-population, that also test positive for the marker. A general strategy for isolating (and counting) the target sub-population, is thus to determine the logical intersection of all sub-populations of cells that test positive for each respective one of the certain set of biochemical markers. In the present instance, an advantageous gating strategy can take account of the following set of properties: the antigen-specific T-cells are a subset of all T-cells; the T-cells are a subset of all lymphocytes; the lymphocytes are a subset of the leukocytes; and the leukocytes are a subset of all blood cells. Accordingly, a suitable gating refinement strategy for isolating the event population corresponding to antigen-specific T-cells will consider progressively narrower subsets of the data in roughly the reverse order. Because such gating is often performed with the aid of two-dimensional bivariate plots (also called dot plots), parameters derived from different detector channels of the cytometer may be considered two-at-a-time in such a refinement.

FIGS. 2A-2C illustrate an example of a gating refinement similar to that described above. FIG. 2A, shows the intensity of CD45 plotted vs. PB/CD3 FITC and further illustrates how a hardware trigger, Tr, represented by the vertical dashed line in FIG. 2A was set on the pacific blue channel (FL6 on the CyAn ADP). In accordance with the gating strategy outlined above, the CD45 marker (plotted on the horizontal axis) is characteristic of leukocytes (as opposed to red blood cells). Thus, the hardware trigger, Tr, which represents a first step in the refinement process, may be set to operate during data acquisition so that most events relating to red blood cells are never recorded. The vertical axis in FIG. 2A represents a detection of a marker, CD3, of the signalling component of the T-cell-receptor (TCR) complex. Using the data plotted in FIG. 2A, regions surrounding the CD3 positive T cells (RI) and the CytoCount beads (R6) were identified and used for gating purposes. Thus, through this type of bivariate dot plot, the population of T-cells, represented by region RI may be isolated. Bivariate plots (dot plots) utilizing either side scatter (SSC) or forward scatter (FSC) may, alternatively, be used in this stage of the analysis. However, it has been found that, as flow rate increases, the precision of scatter-related parameters decreases and, therefore, hardware triggering must be accomplished using a fluorescence parameter

Referring now to FIG. 2B, the refined subset of all the data falling within the region RI was used to gate events for analysis in an MHC Dextramer APC/CD8 PE dot plot. In FIG. 2B, events corresponding to region RI are plotted in a two-dimensional dot plot in which the intensity of events registered in the channel specific for the MHC molecules is plotted as an x-coordinate and the intensity of events registered in the channel specific for the CD8 marker is plotted as a y-coordinate. The marker CD8 is a marker for a co-receptor on cytotoxic T-cells. Thus, in a plot such as is shown in FIG. 2B, the cytotoxic T-cells are isolated from the full population of T-cells and, further, the antigen specific T-cells are isolated from the cytotoxic T-cells. Thus, the CMV antigen specific T cells are located in the upper right quadrant (R3) of FIG. 2B.

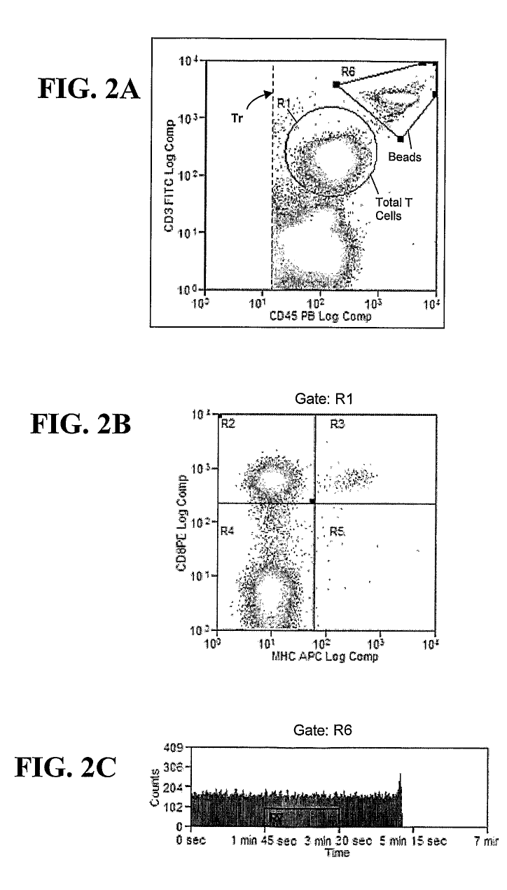

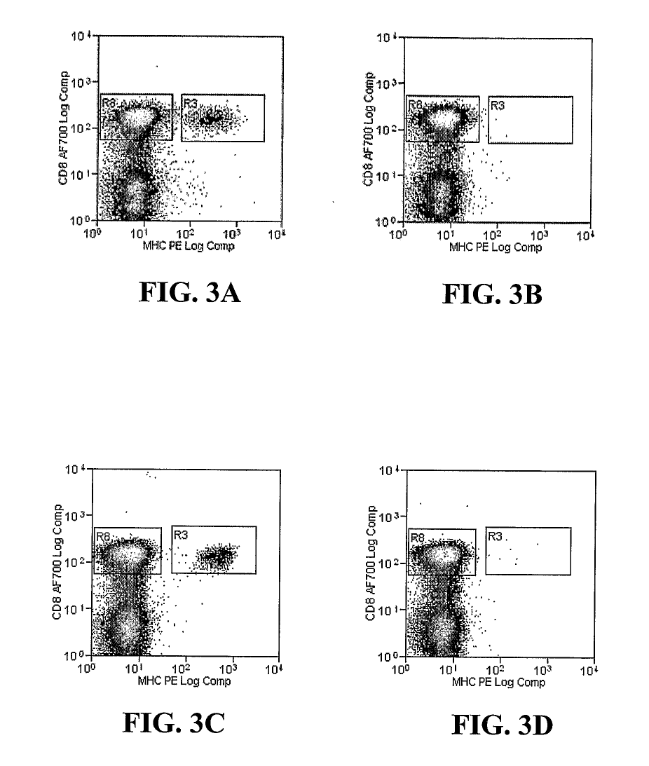

The region R6 (FIG. 2A) was used to gate bead events for analysis in a time histogram. With prior knowledge of the number of beads per total sample volume, the acquisition rate (μL/min) can be calculated from the calculated bead events per minute (FIG. 2C). The number of antigen specific T cells per μL blood can be calculated by comparing the total bead and antigen specific T cell counts when the volume ratio of blood and beads in the sample is known. The stock bead concentration is provided by vendor (Dako). FIG. 2C is a plot of the number of counting beads detected as a function of time. The region R7 is a region that encompasses 1 minute and 45 seconds. Thus one can measure how many bead events there were in that time span allowing for a calculation of the bead event rate. FIGS. 3A-3D are bivariate dot plots of flow-cytometry detection events for a CMV positive whole blood sample from a single donor, wherein the data was obtained using methods of the present invention. The results shown in FIGS. 3A and 3B were obtained in accordance with the method 300 (FIG. 1C) in which both antibody reagents and MHC Dextramer molecules were embedded in a matrix; the results shown in FIGS. 3C and 3D were obtained in accordance with the method 100 (FIG. 1A), in which MHC dextramer reagent and antibody reagents are added to whole blood. FIGS. 3A and 3C show data obtained with a MHC Dextramer molecule loaded with a CMV specific peptide. FIGS. 3B and 3D show data obtained with the same type of MHC Dextramer molecule but loaded with a control peptide. Thus, each of the samples whose data are plotted in FIGS. 3A-3B was run using a pre-packaged container having a matrix within which the antibody reagents or MHC dextramer molecule reagent were held. The matrix was made with 3 mg of Trehalose and 3 mg of Fructose per tube.

The matrix typically comprises 2 microliter (0.2 microgram) antibody per tube, although, in general, a range from 0.02 to 4 microgram of antibody is suitable. The latter amount of antibodies refers to every individual antibody present in the matrix, for example if the matrix comprises a mixture of different antibodies, e.g. anti-CD4 and anti-CD8, each individual antibody of the mixture is present in the indicated amount. MHC dextramer in the matrix was 3 microliter. More generally, MHC dextramer in the matrix may range from 0.3 to 30 microliter per tube (for 100 microliter blood). For the samples for which MHC dextramer was not included in the matrix, the MHC dextramer was applied at 3 microliter per 100 microliter blood. However, a general range of 0.3 to 30 microliter (for 100 microliter blood) is suitable.

The results shown in FIGS. 3A-3D indicate that similar results are obtained between the method 100 and the method 300. However, adding the MHC dextramer reagent separately, instead of providing it retained in or on the matrix, yields better results in our experiment.

The above described method is unique in that it allows for fast sample preparation and fast analysis including acquisition or sorting of cells stained with reagents specific for antigen specific T cells in diluted but otherwise unperturbed blood. The method can be used on any flow cytometer including cell sorters that have fast enough electronics and software. The method can be used for combined staining of both antigen specific structures on the surface of T cells as well as T cell antigens such as CD8 and CD4.

The above disclosed method has been verified experimentally with MHC Class I Dextramer reagents but is expected to function with other types of MHC based reagents including MHC Class II reagents. The MHC reagent may be added firstly followed by a 5 minutes incubation period and subsequent addition of antibody reagents. However, it is also feasible to add premixed MHC- and antibody reagents to the blood. Specifically, it is possible to use premixed fluorochrome labelled Mouse Anti-Human CD8 antibody clone DK25 and MHC reagent. The MHC reagent may be labelled with a fluorochrome such as fluorescein, R-Phycoerythrin (RPE) and allophycocyanin (APC).

It is possible to detect antigen specific T cells with MHC reagent used in combination with a number of antibody reagents such as Anti-Human CD3, CD45 and CD8, for instance Mouse Anti-Human CD3, CD45 and CD8. Furthermore, from these results it is expected that most antibody reagents, including Anti-Human CCR7, CD27, CD28, CD37, CD45RO, CD45RA, CD25, etc., can be used together with MHC reagents.

It is possible to use combinations of MHC reagents with different specificities and/or fluorochrome labels. For instance, it is possible to employ multiple MHC Dextramer types (e.g., different MHC molecules or different peptides loaded into the molecules or both) at the same time. The different reagents could all be labeled similarly as, for instance, when a user wishes to detect the presence of any one of a set of antigen-specific T-cells without regard to the specificity, or with different fluorochromes, as when the user wishes to distinguish among the different types of antigen specificity. Furthermore, it is possible to use such combinations of MHC reagents together with combinations of antibody reagents.

It is also possible to use combinations of MHC reagents, antibody reagents and counting beads in the described assay format. Thus, any combination of MHC-molecule reagents, antibody reagents or counting beads may be employed. This allows for counting the number of cells of various subtypes of antigen specific T cells per volume unit (e.g. μL) of blood or for determining relative values (e.g., percentages) of cell types such as CD3-positive CD8 bright cytotoxic T-cells.

The amount of reagent applied has to be optimised for analysis of whole blood. Much less MHC as well as antibody reagent is used then for a comparable assay where the red blood corpuscles are lysed and the sample washed (lyse/wash). Typically 10-40% MHC reagent and 1-40% antibody reagent is applied for the no-lyse assay compared to a lyse/wash assay.

In comparison with an assay where stained blood is lysed but not washed (lyse/no-wash), the assay described above has the benefit of providing shorter sample handling times and a less harsh environment for the blood cells.

A novel method for high-speed flow cytometry quantification of antigen specific T-cells in whole blood has been disclosed. As can be easily understood from the foregoing, the basic concepts of the present invention may be embodied in a variety of ways. The essence of the invention includes not only sample processing techniques but, also, the various systems, assemblies, and devices required or usable to accomplish the sample processing. Various modifications and variations of the described methods and system of the invention will be apparent to those skilled in the art without departing from the scope and spirit of the invention. Although the invention has been described in connection with specific preferred embodiments, it should be understood that the invention as claimed is not intended to be and should not be unduly limited to such specific embodiments. Indeed, various modifications of the described modes for carrying out the invention which are obvious to those skilled in diagnostic pathology or related fields are intended to be within the scope of the claims.

1. A method for quantification of antigen specific T-cells in un-lysed whole blood, comprising the steps of:
(a) adding an un-lysed whole blood sample to a reaction vessel;
(b) mixing a reagent containing MHC molecules with the un-lysed whole blood;
(c) mixing at least one antibody reagent with the un-lysed whole blood and reagent containing MHC molecules;
(d) incubating the mixture of blood and reagents for a first pre-determined period of time;
(e) diluting the mixture of blood and reagents with an isotonic buffer, in the dilution range of 1:3 to 1:15; and
(f) analyzing the mixture of blood, reagents and isotonic buffer on a flow cytometer analyzer and quantifying antigen specific T-cells present in the whole blood sample.

2. The method of claim 1, further comprising the step of incubating the mixture of whole blood and reagents containing MHC molecules for a second pre-determined period of time, wherein said step follows the step of mixing reagents containing MHC molecules (b).

3. The method of claim 2, wherein the second pre-determined period of time is approximately five minutes.

4. The method of claim 1, wherein the first pre-determined period of time is approximately fifteen minutes.

5. The method of claim 1, wherein the dilution range is 1:10.

6. The method of claim 1, further comprising the step of adding counting beads to the mixture of blood, reagents and isotonic buffer.

7. The method of claim 6, wherein the step of adding counting beads occurs after the diluting step (e).

8. The method of claim 1, wherein the flow cytometer trigger is set to a fluorescence parameter.

9. The method of claim 1, wherein a flow rate through the flow cytometer analyzer is between 50 and 200 μL/min.

10. The method of claim 1, wherein an MHC molecule is labeled with a fluorochrome.

11. The method of claim 1 wherein an antibody is labeled with a fluorochrome.

12. The method of claim 1 wherein an MHC molecule is labeled with a first fluorochrome and an antibody is labeled with a second fluorochrome.

13. The method of claim 1, wherein an MHC molecule comprises a dextran backbone.

14. A method for quantification of antigen specific T cells in un-lysed whole blood, comprising the steps of:
(a) adding at least one antibody reagent to a reaction vessel;
(b) mixing an un-lysed whole blood sample with the at least one antibody reagent;
(c) mixing a reagent containing MHC molecules to the un-lysed whole blood and antibody reagents;
(d) incubating the mixture of blood and reagents for a pre-determined period of time;
(e) diluting the mixture of blood and reagents with an isotonic buffer, in the dilution range of 1:3 to 1:15; and
(f) analyzing the mixture of blood, reagents and isotonic buffer on a flow cytometer analyzer and quantifying antigen specific T-cells present in the whole blood sample.

15. The method of claim 14, wherein the pre-determined period of time is approximately fifteen minutes.

16. The method of claim 14, wherein the dilution range is 1:10.

17. The method of claim 14, further comprising the step of adding counting beads to the at least one antibody reagent, wherein said step follows the step of adding antibody reagents (a).

18. The method of claim 14, wherein the flow cytometer trigger is set to a fluorescence parameter.

19. The method of claim 14, wherein a flow rate through the flow cytometer analyzer is between 50 and 200 μL/min.

20. The method of claim 14, wherein an MHC molecule is labeled with a fluorochrome.

21. The method of claim 14, wherein an antibody is labeled with a fluorochrome.

22. The method of claim 14, wherein an MHC molecule is labelled with a first fluorochrome and an antibody is labeled with a second fluorochrome.

23. The method of claim 14, wherein an MHC molecule comprises a dextran backbone.

24. A kit for preparing an un-lysed whole blood sample for flow cytometric quantification of antigen-specific T cells, said kit comprising:
(a) a container; and
(b) a matrix adhered to at least one wall of the container comprising at least one antibody reagent disposed in or on the matrix, wherein the at least one antibody reagent comprises an antibody capable of binding to a chemical marker characteristic of a particular blood cell type.

25. A kit according to claim 24, further comprising an MHC-molecule reagent disposed in or on the matrix, wherein the MHC-molecule reagent comprises MHC molecules that comprise a peptide that enables binding of the peptide-MHC-molecule complex to the antigen-specific T-cells.

26. A kit according to claim 25 wherein the MHC-molecule reagent is present in or on the matrix in a quantity of from 0.3 to 30 microliter per 100 microliters of un-lysed whole blood sample.

27. A kit according to claim 24, further comprising an MHC-molecule reagent disposed in a second container, wherein the MHC-molecule reagent comprises one or more MHC molecules that comprise a peptide that enables binding of the peptide-MHC-molecule complex to the antigen-specific T-cells.

28. A kit according to claim 25, wherein the MHC molecules comprise a dextran backbone.

29. A kit according to claim 24, further comprising a predetermined quantity of microparticles disposed in or on the matrix.

30. A kit according to claim 29, in which the microparticles comprise polystyrene, latex, agarose or acrylamide beads.

31. A kit according to claim 24, in which the matrix comprises a carbohydrate,

32. A kit according to claim 31, wherein the carbohydrate is a sugar or a mixture of sugars.

33. A kit according to claim 24, wherein the matrix is a polymer or a protein.

34. A kit according to claim 24, wherein each antibody reagent is present in or on the matrix in a quantity of from 0.02 to 4 microgram per 100 microliters of un-lysed whole blood sample.

35. A kit according to claim 24, wherein an antibody is labeled with a fluorochrome.

36. A kit according to claim 24, further comprising instructions for use.

37. A kit for preparing an un-lysed whole blood sample for flow cytometric quantification of antigen-specific T cells, said kit comprising:
(a) a container; and
(b) a matrix adhered to at least one wall of the container comprising at least one MHC-molecule reagent disposed in or on the matrix, wherein the at least one MHC-molecule reagent comprises MHC molecules that comprise a peptide that enables binding of the peptide-MHC-molecule complex to the antigen-specific T-cells.

38. A kit according to claim 37, wherein the matrix further comprises an antibody reagent disposed in a second container, wherein the antibody reagent comprises an antibody capable of binding to a chemical marker characteristic of a particular blood cell type.

39. A kit according to claim 37, wherein the MHC molecules comprise a dextran backbone.

40. A kit according to claim 37, wherein the at least one MHC-molecule reagent is present in or on the matrix in a quantity of from 0.3 to 30 microliter per 100 microliters of un-lysed whole blood sample.

41. A kit according to claim 37, further comprising a predetermined quantity of microparticles disposed in or on the matrix.

42. A kit according to claims 41, in which the microparticles comprise polystyrene, latex, agarose or acrylamide beads.

43. A kit according to claim 37, in which the matrix comprises a carbohydrate.

44. A kit according to claim 43, wherein the carbohydrate is a sugar or a mixture of sugars.

45. A kit according to claim 37, wherein the matrix is a polymer or a protein.

46. A kit according to claim 37, wherein at least one MHC-molecule reagent is labeled with a fluorochrome.

47. A kit according to claim 37, further comprising instructions for use.
